(19) United States
(12) Patent Application Publication
(10) Pub. No.: US 2022/0323672 A1
(43) Pub. Date: Oct. 13, 2022

(54) SYSTEM AND DEVICE FOR DISPENSING A PRODUCT

(71) Applicant: Nemera La Verpillière, LA VERPILLIERE (FR)

(72) Inventors: Loïck Cassagne, Saint-Alban-de-Roche (FR); Pierre Pintus, Crachier (FR); Gaëtan Grenot, Lyon (FR)

(21) Appl. No.: 17/639,852

(22) PCT Filed: Sep. 2, 2020
(86) PCT No.: PCT/EP2020/074498

§ 371 Date: Mar. 2, 2022

(30) Foreign Application Priority Data
Sep. 2, 2019 (FR) FR1909643

(51) Int. Cl. A61M 5/142; A61M 5/145; A61M 5/168

(52) U.S. Cl. CPC A61M 5/14232; A61M 5/1452; A61M 5/16831; A61M 2205/50; A61M 2005/14252

(57) ABSTRACT

A dispensing system includes a reservoir containing the product to be dispensed, a movable element slidably mounted in the reservoir in a sealed manner so as to move according to a level of product in the reservoir, an electric motor, a pump driven by the electric motor and connected to the reservoir to transfer the product from the reservoir to a dispensing device, a first detection member providing information on at least one parameter of the movable element, the first detection member comprising a signal transmitter and a signal receiver,
a second detection member configured to determine at least one operating parameter of the pump or the electric motor,
an electronic member cooperating with the first and second detection members to provide information relating to the dispensing of a predetermined dose of the product.

### FIELD OF THE INVENTION

The invention relates to a system for dispensing a product, in particular a liquid product intended to be introduced in a site of a subject. The invention also relates to a method for using the dispensing system. In addition, the invention relates to a dispensing device comprising the dispensing system.

### BACKGROUND OF THE INVENTION

The state of the art, in particular document WO 2019/008529 A1, already describes a medication dispensing device comprising a reservoir and an optical sensor used to know the volume of liquid dispensed by observing the displacement of a piston in the reservoir. The optical sensor comprises a transmission means located on the piston and a reception means located at the bottom of the reservoir. The signal crosses the reservoir, and therefore the liquid, to provide information relating to the position of the piston. The dispensing system as known in the state of the art presents accuracy defects, since the propagation of the signals transmitted by the transmission means and received by the reception means may be disturbed by the presence of the liquid contained in the reservoir. Determination of the position or displacement of the piston is therefore less accurate, which means that the determination of the quantity of product dispensed is also less accurate. In addition, such an optical sensor cannot detect possible leaks downstream from the reservoir. Sometimes therefore, dispensing of the product may stop although the recommended dose has not yet been fully dispensed. In case of a medicinal product, the treatment may not produce the expected effect, which presents a risk for the patient.

Document WO 2010/069573 A2 which relates to a device for dispensing product contained in a syringe, document WO 02/083209 A1 which relates to a portable pump for dispensing a medication contained in a syringe, document U.S. Pat. No. 6,423,035 B1 which relates to a pump for dispensing a pharmaceutical product contained in a syringe, document WO 2007/062315 A2 which relates to a system for injecting medical fluids and document U.S. Pat. No. 5,662,612 A which relates to a motorised injector are also known.

### SUMMARY OF THE INVENTION

The invention aims in particular to propose a product dispensing system capable of determining more accurately the quantity of product dispensed.

The invention therefore relates to a dispensing system for dispensing a product comprising:
a reservoir of the product to be dispensed,
a movable element slidably mounted in the reservoir in a sealed manner so as to move according to the level of product in the reservoir,
an electric motor,
a pump driven by the electric motor and connected to the reservoir so as to transfer the product from the reservoir to a dispensing means,
a first detection means configured to provide an information on at least one of the parameters of the movable element, selected among the displacement of the movable element in the reservoir, the relative distance of the movable element with respect to a predetermined reference linked to the reservoir and the position of the movable element in the reservoir, the first detection means comprising a signal transmitter and a signal receiver,
a second detection means configured to determine at least one of the operating parameters of the pump or of the electric motor,
an electronic means configured to cooperate with the first detection means and the second detection means to provide information relating to the dispensing of a predetermined dose of product.

The invention therefore proposes to combine two separate detection means to deduce information relating to the dispensing, for example the dispensing or not of a dose of product and/or the dispensing status, for example dispensing in progress or dispensing completed.

In one embodiment, the second detection means is configured to determine at least one of the operating parameters of the pump or of the electric motor, selected among the actuation speed, the actuation duration, an actuation movement and a number of actuation cycles.

“Actuation speed” means, for example, a speed of rotation, forward movement or displacement, etc. Similarly, the term “actuation movement” can refer, for example, to a linear or angular speed or a combination of the two, etc. Lastly, the expression “number of cycles” may correspond, for example, to a number of revolutions, movements, or back and forth movements, etc.

In a preferred configuration, the second detection means is configured to determine at least one of the operating parameters of the pump or of the electric motor, selected among the speed of rotation, the duration of rotation, the angle of rotation and the number of revolutions, preferably selected among the speed of rotation associated with a duration of rotation, the angle of rotation and the number of revolutions.

The volume dispensed can be found by determining the speed of the motor or of the pump, or the number of revolutions made by the motor or by the pump. This information complements the information obtained by the first detection means relating to the displacement or position of the movable element in order to obtain the quantity dispensed more accurately. Knowing the position of the movable element is particularly relevant to identify the moment when this movable element reaches the bottom of the reservoir, and therefore when dispensing of the product is nearly finished. Preferably, operationally, the information relating to the dispensing of a predetermined dose of product can be used to determine the product dose dispensing status, which corresponds to reaching a dispensing rate of between 95% and 100%, preferably between 98% and 100%, of the predetermined dose. The information relating to the dispensing of a predetermined dose of product can also be used to deduce the quantity of product dispensed, continuously or when all the product has been dispensed. The invention allows more reliable determination with better repeatability.

“Product level in the reservoir” means the “quantity of product in the reservoir”. In addition, since the movable element moves according to the level of product in the reservoir, the movable element thus follows the level of liquid in the reservoir, and is not intended to modify the quantity of liquid present in the reservoir. Thus, such a movable element is not the same as the piston of a syringe, for example, which is actuated to act on the level of liquid.

The first detection means may be an optical sensor operating on the Time of Flight (TOF) principle. However, any other type of sensor may be considered, such as a sound or electromagnetic sensor, for example the sensor may be inductive, capacitive, reflective, based on ultrasounds, microwaves, etc. The first detection means can be used to detect the position or displacement of the movable element.

The second detection means may be an encoder for an electric motor, for example optical, magnetic, or electromagnetic. The encoder may be, for example, inductive, capacitive, reflective, based on ultrasounds, microwaves, etc. The encoder can be used to count the number of motor revolutions and then to calculate the volume of product dispensed since a pump is used. For example, when the electric motor makes the pump rotate through one revolution, approximately 1 mL (milliliter) of product is extracted from the reservoir by the pump.

In a preferred embodiment, the pump is a positive displacement pump. Advantageously, the positive displacement pump may be a peristaltic pump.

According to one example, the electronic means may comprise an electronic board. Preferably, the electronic board may comprise a microcontroller and one or more sensors, as well as one or more power components.

The one or more sensors of the electronic board may be inductive, capacitive, reflective, based on ultrasounds, microwaves, etc.

The one or more power components optimize the management of the power allocated to each of the elements present on the electronic board.

According to other optional characteristics of the dispensing system taken alone or in combination:
the movable element is separate from the pump. Thus, the movable element is passive: under the effect of atmospheric pressure, the movable element moves towards the product outlet in the reservoir when the pump dispenses product from the reservoir. In other words, the movable element slides freely in the reservoir. It does not compress the product in the reservoir but moves according to the level of product in the reservoir. Thus, the risk of a product leak at or due to the movable element is reduced and the detection of information relating to the dispensing of product is improved.
the pump is located downstream from the reservoir. In other words, the pump is connected to an outlet of the reservoir and pumps fluid from the reservoir.
the pump is a positive displacement pump, preferably a peristaltic pump, and more preferably a linear or angular peristaltic pump. Thus, the dispensing comfort is improved.
the product is a medicinal product. Thus, the dispensing accuracy is all the more important since the product is a medication.
the signal transmitter and the signal receiver of the first detection means are each arranged in a part of the dispensing system free of product, for example in a part of the reservoir free of product. This arrangement of the signal transmitter and signal receiver allows better signal propagation since this propagation takes place in air and not in the product. The measurement is more accurate and allows the use of any type of product, even opaque. In addition, the detection means can be installed more easily since there is no need to access the inside of the reservoir.

“A part of the dispensing system free of product” means a part which is not in contact with the product. When the signal transmitter and/or the signal receiver is inside the reservoir, it is arranged in a part containing no product; when the signal transmitter and/or the signal receiver is outside the reservoir, it can be attached to the reservoir or arranged on an element that can be detached from the reservoir. Preferably, the signal transmitter and the signal receiver are arranged on a longitudinal axis which is, for example, the longitudinal axis of the reservoir.

When the first detection means is a reflective sensor, the signal transmitter sends a signal to a surface which reflects the signal to the signal receiver, the signal transmitter being arranged near the signal receiver. For example, the signal transmitter and the signal receiver can both be associated with the movable element or the reservoir of the dispensing system. When the first detection means is a sensor without reflection, the signal transmitter sends a signal which is intended to be received directly by the signal receiver, the signal transmitter being arranged opposite to the signal receiver. For example, the signal transmitter can be attached to the movable element and the signal receiver can be attached to the reservoir, or vice versa.
the dispensing system comprises a third detection means configured to determine at least one drive parameter of the pump or of the electric motor, selected among the current intensity of the electric motor and the torque of the pump or of the electric motor, the electronic means being configured to cooperate with the third detection means to provide an information relating to the transfer of the product from the reservoir to the dispensing means. The third detection means provides information in addition to that supplied by the first and second detection means and extends the diagnostic field so as to be able to detect more anomalies. The third detection means may be an optical, sound or electromagnetic sensor, for example the sensor can be inductive, capacitive, reflective, based on ultrasounds, microwaves, etc.

This invention also relates to a method for using a dispensing system as described previously, comprising the following steps:
the first detection means detects the parameter of the movable element, and
the second detection means detects the operating parameter of the pump or of the electric motor,
the electronic means compares the parameter of the movable element with a first predetermined reference, and the operating parameter of the pump or of the electric motor with a second reference to provide an information relating to the dispensing of a predetermined dose of product.

The information relating to the dispensing of a predetermined dose of product may concern in particular the dispensing of the predetermined dose, an occlusion, a leak, a motor problem or a setting fault. It is thus possible to monitor the process of dispensing a product and determine whether a predetermined dose has been completely dispensed. It is also possible to detect anomalies such as a leak or an occlusion between the reservoir and the pump.

Advantageously, the method comprises the following steps:
the third detection means detects the drive parameter of the pump or of the electric motor,
the electronic means compares the drive parameter of the pump or of the electric motor with a third predetermined reference to provide an information relating to the transfer of the product from the reservoir to the dispensing means.

The information relating to the transfer of the product from the reservoir to the dispensing means may concern in particular an occlusion, a leak, a motor problem or a setting fault. As seen above, these determination steps provide information in addition to that supplied by the first and second detection means and extend the diagnostic field so as to be able to detect more anomalies and/or specify the anomalies.

The information relating to the transfer of the product from the reservoir may be communicated to the user via one or more signals using indication means, these indication means possibly being visual indication means, audible indication means or vibratory indication means. One or more signals communicating information relating to the transfer of product from the reservoir could be formed by any combination of the signals transmitted by the visual indication means, the audible indication means or the vibratory indication means.

The invention also relates to a dispensing device for dispensing a product in a site comprising a dispensing system as described previously.

In one embodiment, the dispensing device comprises a housing containing:
the dispensing system as described previously,
a system for inserting a catheter.

Preferably, the system for inserting a catheter comprises a needle movably mounted in the housing, the catheter being connected to the dispensing means of the dispensing system.

Advantageously, the housing comprises two parts:
a first part containing the system for inserting a catheter, and
a second part containing the electric motor.

Preferably, the first part and the second part can be separated.

In a preferred embodiment, the first detection means is placed in the second part of the housing, even more preferably the first detection means comprises a signal transmitter and a signal receiver which are both placed in the second part. Thus, the communication between the signal transmitter and the signal receiver is simplified and does not require additional means of communication of Wi-Fi type.

In a preferred embodiment, the second and third detection means are placed in the second part.

In another embodiment, the signal receiver of the first detection means is placed in the first part.

In another embodiment, the first and second detection means are placed in the first part.

In a preferred embodiment, the pump is contained in the first part of the housing.

According to one embodiment, the first part of the housing is considered to be a disposable part and the second part is considered to be a reusable part. The user can therefore reuse the reusable part comprising the electric motor, with a second disposable part if a second dispensing operation is required, for example if the product is a medication which must be dispensed twice. The disposable part improves compliance with the safety and hygiene constraints.

### BRIEF DESCRIPTION OF THE DRAWINGS

The invention will be better understood on reading the following description, given solely by way of example and with reference to the accompanying drawings in which:

FIG. 1 is a partially transparent top view of a product dispensing system according to one embodiment of the invention;

FIG. 2 is a partially transparent perspective view of the dispensing system of FIG. 1;

FIG. 3a is a schematic view illustrating a first determination provided by a first detection means;

FIG. 3b is a schematic view illustrating a second determination provided by the first detection means of FIG. 3a;

FIG. 3c is a schematic view illustrating a third determination provided by the first detection means of FIG. 3a;

FIG. 4 is a partially transparent perspective view of a product dispensing device comprising the dispensing system of FIG. 1;

FIG. 5a is a schematic view illustrating a first state of the dispensing system of FIG. 1;

FIG. 5b is a schematic view illustrating a second state of the dispensing system of FIG. 1;

FIG. 5c is a schematic view illustrating a third state of the dispensing system of FIG. 1;

FIG. 5d is a schematic view illustrating a fourth state of the dispensing system of FIG. 1.

### DETAILED DESCRIPTION OF THE INVENTION

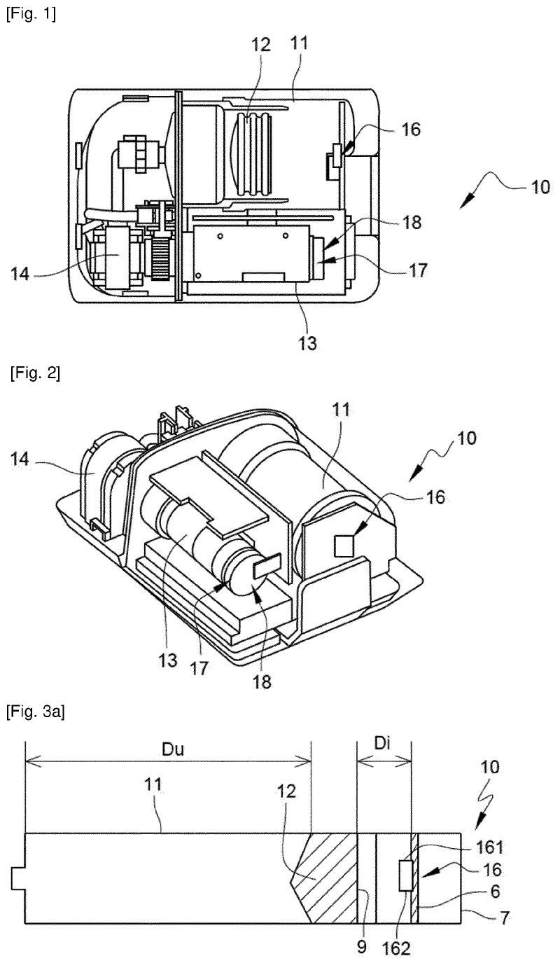

FIGS. 1 and 2 show a system for dispensing product, for example liquid, according to one embodiment of the invention, designated by the general reference 10. The dispensing system 10 comprises a reservoir 11 of the product to be dispensed and a movable element 12 slidably mounted in the reservoir 11 in a sealed manner so as to move according to the level of product in the reservoir 11. In this example illustrated, the reservoir 11 and the movable element 12 have a cylindrical shape. They can have any other suitable shape. The movable element 12 is made of an elastic material, such as rubber, for example, in order to guarantee a better seal with the wall of the reservoir 11.

The dispensing system 10 further comprises an electric motor 13 actuating a pump, in this example a positive displacement pump 14, which is connected to an outlet of the reservoir 11 so as to transfer product from the reservoir 11 to a dispensing means 5 shown on FIG. 4. Under the effect of atmospheric pressure, the movable element 12 moves towards the product outlet of the reservoir 11 when the positive displacement pump 14 pumps product from the reservoir 11. The movable element 12 is separate from the positive displacement pump 14, the positive displacement pump 14 being located downstream from the reservoir 11. For example, the positive displacement pump 14 is a peristaltic pump.

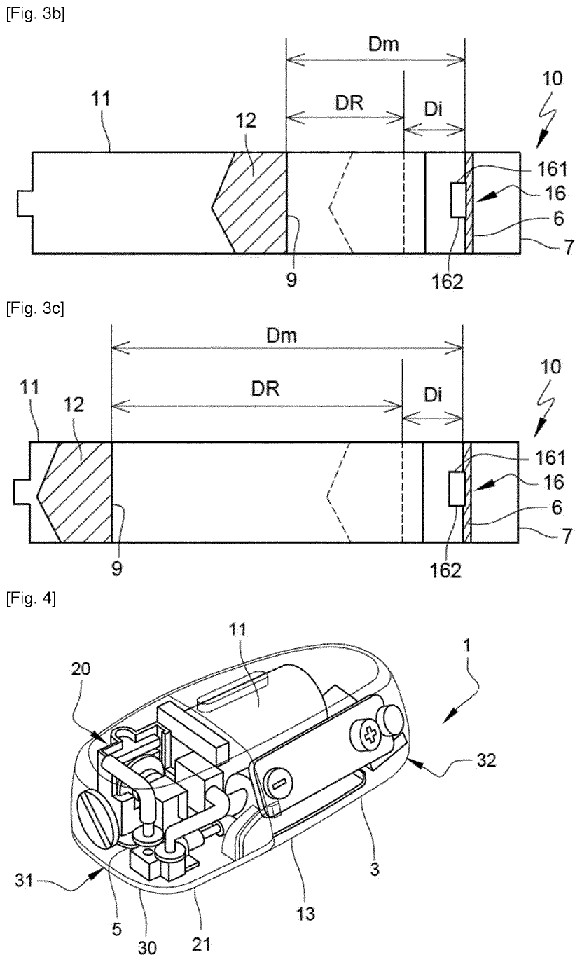

To monitor the displacement of the movable element 12, the dispensing system 10 comprises a first detection means 16 configured to provide information relating to at least one of the parameters of the movable element 12, selected among its displacement, its position and its relative distance from a predetermined reference linked to the reservoir 11. The predetermined reference may for example be one of the ends of the reservoir 11. The first detection means 16 comprises a signal transmitter 161 and a signal receiver 162. According to the example illustrated, the first detection means 16 is an optical sensor 16 capable of determining an absolute distance on the basis of a time of flight of a light beam, therefore independently of the reflectance of the target whose impact is compensated by the optical sensor 16. The operating principle of this first detection means 16 is illustrated on FIGS. 3a and 3b.

Instead of measuring the quantity of light reflected by the object, the optical sensor 16 accurately measures the time required for the light to reach the closest object and return. To do this, the signal transmitter 161 is arranged on a printed circuit board 6 (PCB) carried by a printed circuit board support 7. The assembly of the printed circuit board 6 and the printed circuit board support 7 is placed outside the reservoir 11 and face to the movable element 12. The signal receiver 162 is also arranged on the printed circuit board 6 so that the signal transmitter 161 and the signal receiver 162 are arranged on a longitudinal axis which, in the example illustrated, is the axis of symmetry of the reservoir 11. When the optical sensor 16 is activated, the signal transmitter 161 sends a signal to a surface on the movable element 12 which reflects the signal to the signal receiver 162, the signal being perpendicular to the surface of the movable element 12, thereby allowing the distance between the movable element 12 and the signal transmitter 161 to be calculated as follows: Dm=(C*TOF)/2
Wherein Dm is distance measured between the movable element 12 and the sensor 16;
C is speed of light; and
TOF is time of flight

A reference distance h between the movable element 12 and the sensor 16 is recorded beforehand. This reference distance h may correspond to the length of the reservoir 11. The volume of the reservoir can then be calculated as follows:

V=π*r²*h

wherein V is volume of the reservoir;

r is radius of cross-section of the reservoir; and

h is length of the reservoir

When the optical sensor 16 is placed at the end opposite to the product outlet of the reservoir 11 as illustrated on Fig., the position of the movable element 12 relative to the optical sensor 16 can be compared with the initial reference distance Di. The movable element 12 can move over a useful distance Du to reach the bottom of the reservoir 11. When the actual distance DR moved by the movable element 12 illustrated on FIG. 3B is equal to the useful distance Du, it can be assumed that all the product contained in the reservoir 11 has been dispensed. The actual distance DR moved by the movable element 12 is obtained by subtracting the initial reference distance Di from the measured distance Dm between the movable element 12 and the optical sensor 16.

When the optical sensor 16 is placed at a distance Di from the end opposite to the product outlet of the reservoir 11, as shown on FIGS. 3a and 3b, the volume of the product dispensed can be calculated as a function of the measured distance Dm. For example, when the movable element 12 reaches a position as illustrated on FIG. 3b, the measured dispensed volume is calculated as follows:

VM=π*r²*(Dm−Di)

wherein Vm is measured dispensed volume;

r is radius of cross-section of the reservoir;

Dm is measured distance; and

D is: initial distance.

Depending on the initial distance Di between the optical sensor 16 and the movable element 12, a predetermined reference distance can be recorded. This reference distance may correspond to the length of the reservoir 11 if all the product is to be dispensed in one go; it may also correspond to a partial length if the product contained in the reservoir 11 is to be dispensed in several doses or if only some of the product contained in the reservoir 11 is to be dispensed.

According to another embodiment not shown, the first detection means is arranged to detect the position of the stopper or of the movable element. To this end, the first detection means may comprise a series of sensors arranged along the reservoir and adapted to send a differentiated signal depending on whether or not the stopper is facing their position. Information relating to the position of the stopper and therefore the product dose dispensed can be obtained by analyzing these signals.

According to another embodiment not shown, the first detection means is arranged to detect the movement (or displacement) of the stopper and to differentiate a stationary stopper from a moving stopper. In case of a single-dose dispensing system for which the entire dose is to be administered, detection of the stationary stopper, for example after it has been moving, can be interpreted as meaning that the stopper has reached the bottom of the reservoir, thus providing the information that the product dose to be dispensed has, a priori, been dispensed.

According to another embodiment not shown, the first detection means combines several of the detection modes selected among the distance, the position and the displacement.

In order to make the determinations more accurate, the dispensing system 10 comprises a second detection means 17 (FIG. 1) configured to determine at least one of the operating parameters of the dispensing system 10, more precisely of the electric motor 13 or of the positive displacement pump 14, selected among the speed of rotation, the angle of rotation and the number of revolutions made by at least the positive displacement pump 14 or the electric motor 13.

For a predetermined volume of product to be dispensed, the number of revolutions made by the electric motor 13 or by the positive displacement pump 14 can be calculated as a function of the volume of the positive displacement pump 14 dispensed per revolution. This predetermined number of revolutions is stored in memory. A magnetic encoder can be used on the rear shaft of the electric motor 13 in order to measure the number of revolutions made by the electric motor 13. An optical encoder may also be suitable.

When the measured number of revolutions is equal to the predetermined number of revolutions, it can be assumed that the entire predetermined volume of the product has been dispensed.

The dispensing system 10 comprises an electronic means 4, for example an electronic board, configured to cooperate with the first detection means 16 and the second detection means 17 to provide an information relating to the dispensing of a predetermined dose of product.

In order to make the determinations even more accurate, the dispensing system 10 may comprise a third detection means 18 configured to determine at least one of the drive parameters of the dispensing system 10, more precisely of the electric motor 13 or of the positive displacement pump 14, selected among the current intensity of the electric motor 13, and the torque of at least the positive displacement pump 14 or of the electric motor 13, the electronic means 4 being configured to cooperate with the third detection means 18 to provide an information relating to the transfer of the product from the reservoir 11 to the dispensing means 5.

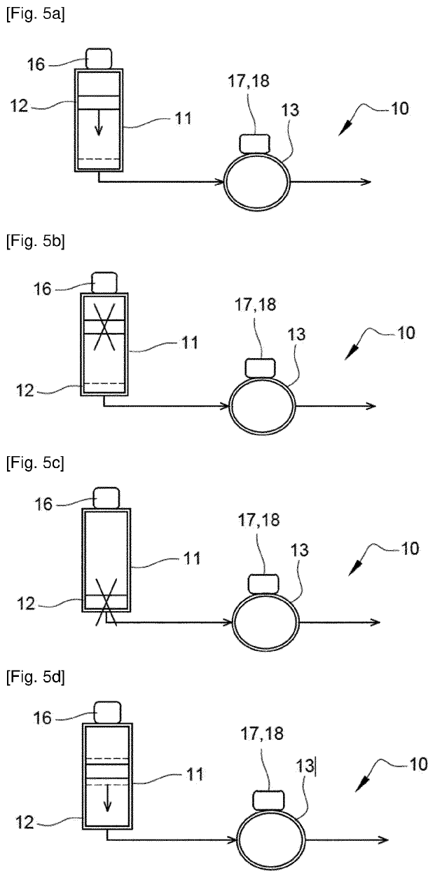

FIGS. 5a to 5d are schematic representations of several configurations of use of the dispensing system 10 with a first detection means 16, a second detection means 17 and/or a third detection means 18. The displacement of the movable element 12 is indicated by an arrow on the movable element 12. The non-displacement of the movable element 12 is indicated by a cross on the movable element 12. The theoretical position of the movable element 12 having reached the predetermined distance is indicated by dotted lines. The flow of the product, when it is extracted from the reservoir 11, follows the direction of the arrows. The first detection means 16 detects a parameter of the movable element 12 which may be the measured distance Dm between the movable element 12 and the sensor 16; the second detection means 17 detects an operating parameter of the dispensing system 10 which may be the number of revolutions made by the electric motor 13; the third detection means 18 detects a drive parameter of the dispensing system 10 which may be the current intensity of the electric motor 13, the electronic means 4 compares the parameter of the movable element 12 with a first predetermined reference which may be the predetermined distance, the operating parameter of the dispensing system 10 with a second reference which may be the predetermined number of revolutions made by the electric motor 13, and the drive parameter of the dispensing system 10 with a third reference which may be a predetermined current intensity of the electric motor 13.

To simplify the following description, the expression “the number of revolutions” is used in a non-limiting manner. The predetermined number of revolutions can be replaced by an angle of rotation, a speed of rotation associated with a duration of rotation. Similarly, the expression “current” is used in a non-limiting manner. The current can be replaced by the torque of the positive displacement pump 14 or the torque of the electric motor 13.

The following description mainly concerns a device for dispensing an incompressible liquid product with the resulting deductions of the electronic means 4. Interpretation variants for compressible products are also provided.

As shown on FIG. 5a, in a first state, the movable element 12 is detected as being moving and the predetermined distance is detected as not being reached, or not detected.

According to a first configuration of FIG. 5a, if the predetermined number of revolutions made by the electric motor 13 is detected as not being reached, or not detected, and if the predetermined current intensity is detected as not being reached, or not detected, the electronic means 4 deduces that the predetermined dose is being dispensed.

According to a second configuration of FIG. 5a, if the predetermined number of revolutions made by the electric motor 13 is detected as not being reached, or not detected, and if the predetermined current intensity is detected as being reached, the electronic means 4 deduces that the dispensing system 10 has encountered at least a fault of the electric motor 13 and possibly a problem concerning the configuration of at least one of the first, second and third predetermined references. On a positive displacement pump, for example, in normal operation the current intensity of the electric motor varies very slightly around a given value. If the current intensity value starts to increase significantly, this means that the motor is forcing in reaction to a resistance. The resistance is due to a product transfer problem such as an occlusion, or to a motor fault. For a liquid product, any occlusion will cause the movable element 12 to stop. Thus, in case of an abnormal current intensity, if the movable element 12 is stationary it is possible to conclude for an incompressible product that the electric motor has a fault and that there may also be a configuration problem.

According to a third configuration of FIG. 5a, if the predetermined number of revolutions made by the electric motor 13 is detected as being reached, and if the predetermined current intensity is detected as not being reached in a non-lasting manner, for example of the order of less than one second, or not detected, the electronic means 4 deduces that the dispensing of the predetermined dose is, a priori, nearly finished.

According to a fourth configuration of FIG. 5a, if the predetermined number of revolutions made by the electric motor 13 is detected as being reached, and if the predetermined current intensity is detected as being reached, the electronic means 4 makes a deduction similar to that of the second configuration.

According to a fifth configuration of FIG. 5a, if the predetermined number of revolutions made by the electric motor 13 is detected as being reached, and if the predetermined current intensity is detected as not being reached in a lasting manner, for example of the order of a few seconds, the electronic means 4 deduces that the dispensing system 10 has encountered a problem concerning the configuration of at least one of the first, second and third predetermined references.

As shown on FIG. 5b, in a second state, the movable element 12 is detected as being stationary and the predetermined distance is detected as not being reached.

According to a first configuration of FIG. 5b, if the predetermined current intensity is detected as not being reached, or not detected, and if the predetermined number of revolutions made by the electric motor 13 is detected as not being reached, the electronic means 4 deduces that the dispensing system 10 has encountered a problem concerning an occlusion upstream from the positive displacement pump 14 or a leak upstream from the positive displacement pump 14.

According to a second configuration of FIG. 5b, if the predetermined number of revolutions made by the electric motor 13 is detected as not being reached, and if the predetermined current intensity is detected as being reached, the electronic means 4 deduces that the dispensing system 10 has encountered at least a problem concerning an occlusion downstream from the positive displacement pump 14 and possibly a fault of the electric motor 13 and possibly a problem concerning the configuration of at least one of the first, second and third predetermined references.

According to a third configuration of FIG. 5b, if the predetermined number of revolutions made by the electric motor 13 is detected as being reached, and if the predetermined current intensity is detected as not being reached in a non-lasting manner, for example of the order of less than one second, the electronic means 4 deduces that the dispensing of the predetermined dose is, a priori, nearly finished.

According to a fourth configuration of FIG. 5b, if the predetermined number of revolutions made by the electric motor 13 is detected as being reached, and if the predetermined current intensity is detected as being reached, the electronic means 4 deduces that the dispensing system 10 has encountered at least a problem concerning an occlusion downstream from the positive displacement pump 14 and possibly a fault of the electric motor 13 and possibly a problem concerning the configuration of at least one of the first, second and third predetermined references.

According to a fifth configuration of FIG. 5b, if the predetermined number of revolutions made by the electric motor 13 is detected as being reached, and if the predetermined current intensity is detected as not being reached in a lasting manner, for example of the order of a few seconds, the electronic means 4 deduces that the dispensing system 10 has encountered a problem concerning the configuration of at least one of the first, second and third predetermined references.

As shown on FIG. 5c, the movable element 12 is detected as being stationary and the predetermined distance is detected as being reached or not detected.

According to a first configuration of FIG. 5c, if the predetermined number of revolutions made by the electric motor 13 is detected as not being reached, or not detected, and if the predetermined current intensity is detected as not being reached in a non-lasting manner, for example of the order of one second, or not detected, the electronic means 4 deduces that the dispensing of the predetermined dose is nearly finished.

According to a second configuration of FIG. 5c, if the predetermined number of revolutions made by the electric motor 13 is detected as not being reached, or not detected, and if the predetermined current intensity is detected as being reached, the electronic means 4 deduces that the dispensing system 10 has encountered at least a problem concerning an occlusion downstream from the positive displacement pump 14 and possibly a fault of the electric motor 13 and possibly a problem concerning the configuration of at least one of the first, second and third predetermined references.

According to a third configuration of FIG. 5c, if the predetermined number of revolutions made by the electric motor 13 is detected as being reached, and if the predetermined current intensity is detected as not being reached, or not detected, the electronic means 4 deduces that the predetermined dose is, a priori, being dispensed.

According to a fourth configuration of FIG. 5c, if the predetermined number of revolutions made by the electric motor 13 is detected as being reached, and if the predetermined current intensity is detected as being reached, the electronic means 4 deduces that the dispensing system 10 has encountered at least a problem concerning an occlusion downstream from the positive displacement pump 14 and possibly a fault of the electric motor 13 and possibly a problem concerning the configuration of at least one of the first, second and third predetermined references.

According to a fifth configuration of FIG. 5c, if the predetermined number of revolutions made by the electric motor 13 is detected as not being reached, and if the predetermined current intensity is detected as not being reached in a lasting manner, for example of the order of a few seconds, the electronic means 4 deduces that the dispensing system 10 has encountered a problem concerning the configuration of at least one of the first, second and third predetermined references.

As shown on FIG. 5d, in a fourth state, the movable element 12 is detected as moving and the predetermined distance is detected as being reached.

According to a first configuration of FIG. 5d, if the predetermined number of revolutions made by the electric motor 13 is detected as not being reached, or not detected, and if the predetermined current intensity is detected as not being reached, or not detected, the electronic means 4 deduces that the dispensing of the predetermined dose is nearly finished.

According to a second configuration of FIG. 5d, if the predetermined number of revolutions made by the electric motor 13 is detected as not being reached, or not detected, and if the predetermined current intensity is detected as being reached, the electronic means 4 deduces that the dispensing system 10 has encountered at least a fault of the electric motor 13 and possibly a problem concerning the configuration of at least one of the first, second and third predetermined references.

According to a third configuration of FIG. 5d, if the predetermined number of revolutions made by the electric motor 13 is detected as being reached, and if the predetermined current intensity is detected as not being reached, or not detected, the electronic means 4 deduces that the predetermined dose is, a priori, being dispensed.

According to a fourth configuration of FIG. 5d, if the predetermined number of revolutions made by the electric motor 13 is detected as being reached, and if the predetermined current intensity is detected as being reached, the electronic means 4 deduces that the dispensing system 10 has encountered at least a fault of the electric motor 13 and possibly a problem concerning the configuration of at least one of the first, second and third predetermined references.

The operating states of the dispensing system 10 are not limited to those described. In the example shown, the dispensing system 10 is single-dose type, in other words designed to dispense the entire dose continuously in one go. The dispensing system may also be designed so that not all the product contained in the reservoir 11 is dispensed. The dispensing system may also be of multi-dose type, in other words doses are administered successively in a non-continuous manner, the doses possibly being equivalent or not in terms of flow rate and/or volume of product dispensed.

FIG. 4 illustrates a device 1 for dispensing a product in a site of a subject comprising a housing 2 containing a dispensing system 10 as described previously and a system 20 for inserting a catheter 30. The insertion system 20 further comprises a needle 21 movably mounted in the housing 2. The catheter 30 is connected to the dispensing means of the dispensing system 10.

According to one embodiment of the dispensing device 1, the housing 2 comprises a first part 31 containing the system 20 for inserting the catheter 30, the reservoir 11 and the positive displacement pump 14 and a second part 32 containing the electric motor 13. According to this embodiment, the first part 11 and the second part 12 can be removed or separated from each another. According to another configuration, the pump 14 can be included in the second part 32 of the housing 2.

The invention is not limited to the embodiments described and other embodiments will be clearly apparent to those skilled in the art. In particular, the dispensing system or the dispensing device may comprise means for indicating information relating to the dispensing of a determined dose of product, controlled by the electronic means 4.

The movable element may also form a part of the reservoir, for example the wall of a flexible pouch.

1. A dispensing system for dispensing a product comprising:
a reservoir of the product to be dispensed,
a movable element slidably mounted in the reservoir in a sealed manner so as to move according to a level of the product in the reservoir,
an electric motor,
a pump driven by the electric motor and connected to the reservoir so as to transfer the product from the reservoir to a dispensing device,
a first detection member configured to provider information on at least one parameter of the movable element, selected from a displacement of the movable element in the reservoir, a relative distance of the movable element with respect to a predetermined reference linked to the reservoir, and a position of the movable element in the reservoir, the first detection member comprising a signal transmitter and a signal receiver,
a second detection member configured to determine at least one operating parameter of the pump or of the electric motor,
an electronic member configured to cooperate with the first detection member and the second detection member to provide information relating to the dispensing of a predetermined dose of the product.

2. The dispensing system according to claim 1, wherein the at least one operating parameter of the pump or of the electric motor is selected from an actuation speed, an actuation duration, an actuation movement and a number of actuation cycles.

3. The dispensing system according to claim 1, wherein the pump is a positive displacement pump, and wherein the at least one operating parameter of the pump or of the electric motor, is selected from a speed of rotation, a duration of rotation, an angle of rotation and a number of revolutions.

4. The dispensing system according to claim 1, wherein the movable element is separate from the pump.

5. The dispensing system according to claim 1, wherein the pump is located downstream from the reservoir.

6. The dispensing system according to claim 1, wherein the pump is a peristaltic pump.

7. The dispensing system according to claim 1, wherein the signal transmitter and the signal receiver of the first detection member are each arranged in a part of the dispensing system that is free of the product.

8. The dispensing system according to claim 1, comprising a third detection member configured to determine at least one drive parameter of the pump or of the electric motor, selected from a current intensity of the electric motor and a torque of the pump or of the electric motor, wherein the electronic member is configured to cooperate with the third detection member to provide information relating to the transfer of the product from the reservoir to the dispensing device.

9. A method for using the dispensing system of claim 1, comprising the following steps:
the first detection member detects the at least one parameter of the movable element, and
the second detection member detects the at least one operating parameter of the pump or of the electric motor, and
the electronic member compares the at least one parameter of the movable element with a first predetermined reference, and the at least one operating parameter of the pump or of the electric motor with a second reference to provide information relating to the dispensing of a predetermined dose of the product.

10. The method for using the dispensing system according to claim 9, wherein the dispensing system further comprises a third detection member configured to determine at least one drive parameter of the pump or of the electric motor selected from a current intensity of the electric motor and a torque of the pump or of the electric motor, wherein the electronic member is configured to cooperate with the third detection member to provide information relating to the transfer of the product from the reservoir to the dispensing device, the method further comprising the following steps:
the third detection member detects the at least one driver parameter of the pump or of the electric motor,
the electronic member compares the at least one drive parameter of the pump or of the electric motor with a third predetermined reference to provide information relating to the transfer of the product from the reservoir to the dispensing device.

11. A dispensing device for dispensing a product in a site, comprising the dispensing system according to claim 1.

12. The dispensing device according to claim 11, comprising a housing containing:
the dispensing system, and
a system for inserting a catheter comprising a needle movably mounted in the housing, the catheter being connected to the dispensing device of the dispensing system,
the housing comprising
a first part containing the system for inserting a catheter, and
a second part containing the electric motor.
